(19) United States
(12) Patent Application Publication
(10) Pub. No.: US 2021/0379164 A1
(43) Pub. Date: Dec. 9, 2021

(54) ENZYMES AND METHODS FOR PREVENTING AND TREATING PYRETHROID EXPOSURE IN ANIMALS

(71) Applicant: Ecto Development Corporation, Blue Springs, MO (US)

(72) Inventors: Bobby Rose, Blue Springs, MO (US); Rich Rose, Blue Springs, MO (US); Brian Compton, Pleasant Hill, MO (US); Ashley Siegel, St. Louis, MO (US); Alejandro Tovar-Mendez, Columbia, MO (US)

(21) Appl. No.: 17/237,193

(22) Filed: Apr. 22, 2021

Related U.S. Application Data
(63) Continuation of application No. 16/892,914, filed on Jun. 4, 2020, now abandoned.

(51) Int. Cl. A61K 38/46

(52) U.S. Cl. CPC A61K 38/465; C12Y 301/01001

(57) ABSTRACT

Compositions and methods for treating an animal who has been exposed to pyrethroid are provided. An effective amount of a pyrethroid detoxifying enzyme to prevent or treat pyrethroid toxicity. The pyrethroid detoxifying enzyme is one or more pyrethroid-hydrolyzing carboxylesterases.

### BACKGROUND

Pyrethroids or synthetic pyrethroids have insecticidal activity and target sodium channels in nervous system tissues. Pyrethroids constitute the majority of commercial household insecticides. Pyrethroids are effective and widely used insecticides found in many common pesticide products for control of general pests such as mosquitoes, fleas, ticks, ants, spiders, cockroaches, mites and agricultural pests. Pyrethroids are esters of dibromo- or dicloro-vinyl cyclopropane carboxylate. Exemplary pyrethroids include pyrethrin and its derivatives, fenpropathrin, bifenthrin, cyfluthrin, beta-cylfuthrin, pyrethrin, phenothrin, imiprothrin, flumethrin, momfluorothrin, bioallethirn, deltamethrin, cypermethrin, tetramethrin, allethrin, phenothrin, barthrin, dimethrin, bioresmethrin and permethrin, a dichlorovinyl derivative of pyrethrin, the most widely used pyrethroid. Permethrin is a common insecticide used as an insect repellant for medical use in humans and animals and as a residential and commercial pesticide. Pesticide-grade pyrethroids, such as permethrin, while relatively safe to use for humans and dogs, are toxic to cats and aquatic animals. Many cats die after being given flea treatments intended for dogs, or by contact with dogs having recently been treated with permethrin. Signs of exposure to permethrin in cats include hyperthermia, hyperesthesia, hyper salivation, convulsions and in some instances death.

Pyrethroid Neurotoxicosis is a known problem in veterinary applications to the point that the EPA has restricted uses on cats, requiring special notations, verbiage labeling requirements and registration processes. This invention may prevent or reduce these requirements which would save EPA and registrants time and money. Cats preen themselves to remove contaminants on their fur and may accidentally poison themselves if the cat has pyrethroids on their fur from veterinary applications. This invention degrades pyrethroids and in turn reduces toxicosis.

### SUMMARY

Embodiments of the present invention relate to administering an effective amount of hydrolyzing carboxylesterase to an animal to prevent or reduce the toxic effects of permethrin and synthetic pyrethroids, particularly felines because of their sensitivity to this family of insecticides due to a deficiency of a hepatic glucuronosyltransferase enzyme.

Embodiments of the present invention are directed to an end use product that contains an enzyme that can be used to prevent (vaccination) or treat (antidote) synthetic pyrethroid toxicity through injection, ingestion, or topical medication. Multiple enzymes were produced (via genetically modified bacteria), synthesized, analyzed and developed.

Effectiveness of the enzymatic solutions were analyzed by a HPLC permethrin degradation byproduct assay (permethrin byproduct is PBA), bioassay, and HPLC analysis in blood serum to confirm and verify the degradation of permethrin by enzymes, particularly PytH2

A standard of permethrin (CAS Number 9016-189-6) was prepared and analyzed by HPLC as a control, then different concentrations of enzymatic solution were added to the standard to find an effective concentration (of pPytH2) needed to degrade the permethrin. After degradation was confirmed through HPLC analysis by the qualification and quantitation of permethrin hydrolysis byproducts, a bioassay was performed with crustaceans (scuds), who are also highly sensitive to pyrethroid toxicity. In the bioassay, scuds were exposed to two different environments. Permethrin was added to the first environment to get a scud kill count without the presence of the enzyme as a control and the next environment was evaluated by adding both permethrin and the degrading enzyme to the scud environment and kill counts were compared.

The bioassay showed that PytH2 protein hydrolyzed the toxic permethrin molecule and degraded permethrin. The next step was to test the enzymatic hydrolysis of permethrin in feline blood (cat serum). Cat serum was acquired, permethrin was added to the serum and was assayed as a control by HPLC. After the cat serum control was analyzed three different concentrations of the enzyme were introduced to the cat serum control and then analyzed by HPLC. The results of the analysis show that 10 μl enzyme concentration added to the poisoned cat serum degraded all of the permethrin and the HPLC analyzed 0.0% permethrin in the control cat serum after the enzyme was applied.

In another embodiment, the PytH2, enzyme degrades other synthetic pyrethroids in addition to permethrin, specifically cypermethrin, deltamethrin, and fenpropathrin, through biological and analytical assays. Multiple synthetic enzymes were tested and shown that the PytH2 degrades at least four synthetic pyrethroids that are commonly used in the animal field.

These methods are providing an injectable enzyme solution in animals, such as rats, felines or other animals that have been poisoned by permethrin or synthetic pyrethroids.

### BRIEF DESCRIPTION OF THE DRAWINGS

Illustrative aspects of the present invention are described in detail below with reference to the attached drawing figures, and wherein:

FIG. 1 is graph of enzymes expressed with a tag (e.g., a polyhistidine (His)) to facilitate purification.

FIG. 2 is a graph of absorbance.

FIGS. 3 and 4 are graphs of a permethrin standard curve.

FIG. 5 is a graph of deltamethrin reaction with PYTH2.

FIG. 6 is a graph of estherase activity.

FIG. 7 is graph of hydrolysis of trans permethrin.

FIGS. 8 and 9 are graphs of scuds exposed to permethrin and esterases.

FIG. 10 is a graph of a nucleotide amplicon.

FIG. 11 is a graph of a plasmid.

### DETAILED DESCRIPTION

The present invention is generally directed as a method for administering an effective amount of detoxifying enzymes and compositions to an animal to prevent or treat pyrethroid toxicity in animals. The present invention is also directed to treating animals with permethrin exposure.

The enzymes can be used in methods for detoxifying pyrethroids. The present invention relates to proteins having pyrethroid degradation abilities (esterase) including proteins containing a targeting sequence having protease properties.

An esterase is a hydrolase that splits esters into acids and alcohols. An esterase can act as a pyrethroid hydrolase, hydrolyzing permethrin into (3-phenoxyphenyl) methanol and (1S,3R)-3-(2,2-dichloroethenyl)-2,2 dimethylcyclopropanecarboxylate. Enzymes were purified from an E. coli strain optimized for recombinant protein expression.

The specific reason for the sensitivity of the cat is also unknown. Species susceptibility to permethrin is likely dependent on the nature of the tissue esterase, the level of activity detected, the substrate specificity, and the rate of hydrolysis encountered. Since hydrolytic enzymes degrade pyrethroid esters, it is suspected that the species susceptibility of permethrin could be due to the rate of hydrolysis being slower in cats than other species. Cats naturally lack the production of glucuronidyltransferase in the liver and this invention would provide an enzyme that would metabolize the pyrethroid to prevent toxicosis.

### Detoxifying Enzymes

As described in greater detail below, the genomes of microbes in microbial libraries that contained microorganisms capable of degrading pyrethroid, particularly pyrethrin, were screened to identify enzymes capable of degrading pyrethroids. This screening resulted in identification of a number of pyrethroid-hydrolyzing carboxylesterases.

Pyrethroid-hydrolyzing carboxylesterases can be used to detoxify pyrethroids and degrade pyrethroids in animals. For example, pyrethroid-hydrolyzing carboxylesterases can be used to promote accelerated degradation of pyrethroid, as compared to the rate of degradation that would occur in the absence of the esterase. The mechanism for detoxifying pyrethroid by an esterase is shown 1) hydrolysis of ester bound between dibromo- or dichloro-vinyl cyclopropane carboxylate.

A reaction scheme for detoxification by esterase is shown below:

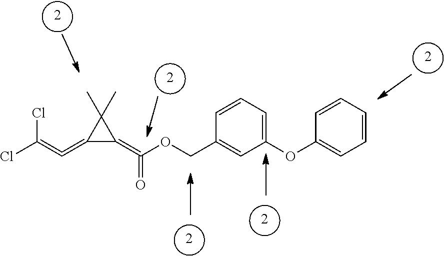

Selected esterase activity for Permethrin degradation

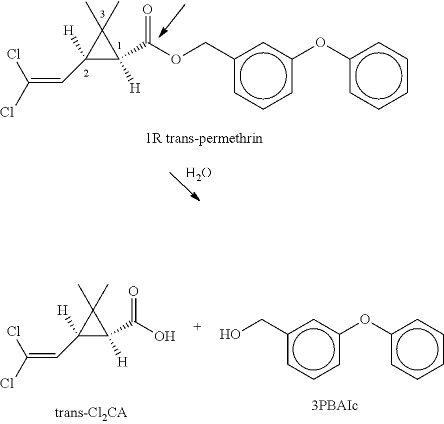

### Initial Screen

Initial screen to identify esterase candidates was performed. The initial screen was performed by the ability to grow in the presence of permethrin 0.2% on a solid plate. Over 30 expected tolerant strains were tested. The next step was to look for liquid growth of the identified candidates

Permethrin was not soluble in water and once dissolved in a carrier such as methanol, DMSO, or xylene it was challenging to keep the chemical in solution when diluted into a media appropriate for bacterial growth. Thus, a six hour growth assay with BHI and 0.2% Permethrin dissolved in methanol was used to screen the same strains that were initially tolerant. This assay was performed three times to look for consistency in performance.

Candidates were moved to a minimalist media to force the bacteria to use the permethrin as a carbon source. Bacterial CFU at 0, 6, 24, 30, 48 and 72 hours were collected to assess growth. This assay was performed three times. At this point five (5) candidates emerged as possible degrading strains of permethrin. Those candidate strains were then screened using pyrethrin as the sole carbon source to look for general pyrethroid degrading ability

Correct controls were created for the newly created bacterial growth assay as assessment for degradation of chemicals. At this point, one of the five microbial candidate enzymes was identified as a specific carboxylesterase for degradation in plasma. This enzyme was cloned into an expression vector system so a Spectral Analysis of the degradation of permethrin by the enzyme over time in a cell free assay could be performed.

The five enzymes having pyrethroid degradation abilities are identified in Table 1.

TABLE 1

| Abbre- | | | Amino Acid | DNA SEQ |
|---|---|---|---|---|
| viation | Esterase | Source | SEQ ID No. | ID No. |
| E1 | pytZ | Ochrobactrum sp. | SEQ. ID No. 1 | |
| E2 | pnbA | B. subtilis | SEQ. ID No. 2 | |
| E3 | PytH2 | Sphingobium | SEQ. ID No. 3 | SEQ. ID No. 6 |
| | (pytH) | wenxiniae | | |
| E4 | MsE1 | Methylobacterium | SEQ. ID No. 4 | |
| | | sp. | | |
| E5 | EstP | Klebsiella sp. | SEQ. ID No. 5 | |

With reference to nucleic acids of the invention, the term “isolated nucleic acid” is sometimes used. This term, when applied to DNA, refers to a DNA molecule that is separated from sequences with which it is immediately contiguous (in the 5′ and 3′ directions) in the naturally occurring genome of the organism from which it originates. For example, the “isolated nucleic acid” may comprise a DNA or cDNA molecule inserted into a vector, such as a plasmid or virus vector, or integrated into the DNA of a prokaryote or eukaryote.

With respect to protein, the term “isolated protein” or “isolated and purified protein” is sometimes used herein. This term refers primarily to a protein produced by expression of an isolated nucleic acid molecule of the invention. Alternatively, this term may refer to a protein which has been sufficiently separated from other proteins with which it would naturally be associated, so as to exist in “substantially pure” form.

The term “vector” refers to a small carrier DNA molecule into which a DNA sequence can be inserted for introduction into a host cell where it will be replicated. An “expression vector” is a specialized vector that contains a gene or nucleic acid sequence with the necessary regulatory regions needed for expression in a host cell.

For ease of reference, amino acid sequences of the enzymes are provided in Table 2.

TABLE 2

| Esterase name and source | Amino Acid Sequence |
|---|---|
| EstP | MEICTKGSRKHLTSRASEPSYNVPENQYVLYVVSSTLSIVICQLVKV |
| (Klebsiella sp.) | AESKKSRFSGAIEKLNERLDSLKDYRIINRDLVVKDLERLKKRFDT |
| SEQ. ID No. 5 | EVINAELSEQLAKINVNLSRSYSEKGYLRLEKATGSENEWWAK1K |
| | PSQNDDWQQFEPDGYTIFSSRDHYASLEKSYSDYEIEAKIPLPLRR |
| | GICAVVLYPEYISRICVLPESRSIQREQENFTKLRDKGIALSKKDWQ |
| | AKLTIDELAEQEKERATINKRLGYFATEHEKVGIVHKALEPKLICP |
| | FQQIEKEWRQCKVKSKSTFPNSMTFVQNPAYQAVHSGFKKLKEQI |
| | GLADEDILLSLEKIEAIGLVNMPLLYERWCLLQIIKVLTQAFRYQPE |
| | DNWKRKLIANIQGNEEQISIQFFNPSVSRAITLQYEPFLANGKRPDF |
| | VLDVEAITKSGNQISKRLVVDAKYYSAAYLKQRGGIGGVIHELYN |
| | GKDYSECQENSVFVLHPVLDAVEKVVSPQEWAKDSYLGELSMFD |
| | WEPAHHQRQATNYGAVCANPMKSQRYLDEIQRMLGMFLQYGIE |
| | DNTSFRGASDDTHAVNFCVSCGSEKVVDVTKSMSSNNQKRWYR |
| | CNECTHFTVYTHCGTCNTRLIKNGEYWTYLSLMPMSSINIKCPNC |
| | ESPV |
| MsE I | MTQDTTGFIFIREEPGPDTTRPLMLLHGTGGDENDLLPLGRMVAPE |
| (Methylobacterium sp.) | AALLAPRGGVSENGMPRFFRRLAEGVFDEADLRRRTGDLAAFVA |
| SEQ. ID. No. 4 | ASRARYGLGAPLALGFSNGANIAASLLMLRPETLTGAVLIRPMVPF |
| | AEPPAADLAGRPVLILSGAMDPIVPVENARRLAQQLSASGARVEH |
| | RILPAGHGLSQADVSQALAWLRSLPGPEAA |
| PnbA | MTHQIVTTQYGKVKGTTENGVHKWKGIPYAKPPVGQWRFKAPEP |
| (Bacillus subtilis) | PEVWEDVLDATAYGSICPQPSDLLSLSYTELPRQSEDCLYVNVFAP |
| SEQ. ID. No. 2 | DTPSKNLPVMVWIHGGAFYLGAGSEPLYDGSKLAAQGEVIVVTL |
| | NYRLGPFGELHLSSFNEAYSDNLGLLDQAAALKWVRENISAFGGD |
| | PDNVTVFGESAGGMSIAALLAMPAAKGLFQICAIMESGASRTMTIC |
| | EQAASTSAAFLQVLGINEGQLDKLHTVSAEDLLKAADQLRIAEKE |
| | N1FQLFFQPALDPKTLPEEPEKAIAEGAASGIPLLIGTTRDEGYLEFT |
| | PDSDVHSQETLDAALEYLLGKPLAEKVADLYPRSLESQIHMMTDL |
| | LEWRPAVAYASAQSHYAPVWMYREDWHPKKPPYNKAFHALELP |
| | FVFGNLDGLERMAKAEITDEVKQLSHTIQSAWITFAKTGNPSTEA |
| | VNWPAYHEETRETLILDSEITIENDPESEICRQKLEPSKGE |
| pPytH2 | MTVTDIILIHGALNRGACYDAVVPLLEARGYRVHAPDLTGHTPGD |
| (Sphingobium wenxiniae) | GGHLSVVDMEHYTRPVADILARAEGQSILLGHSLGGASISWLAQH |
| SEQ. ID. No. 3 | HYDKVAGLIYLTAVLTAPGITPETFVLPGEPNRGTPHALDLIQPVDE |
| | GRGLQADFSRLERLREVFMGDYPGEGMPPAEQFIQTQSTVPFGTP |
| | NPMEGRALEIPRLY1EALDDVVIPIAVQRQMQKEFPGPVAVVSLPA |
| | SHAPYYSMPERLAEAIADFADAPAEYRQTATKAGPDRPAGADGG |
| | RADRADLP |
| PytZ | MTTQTYEHRLKAGAKGAPLFIVEHGTGGDENQFFGLAEQLLPDAT |
| (Ochrobactrum sp.) | IMSPRGDVSEYGAARFFRRTGEGVYDMEDLARATDKMAGFIAAL |
| SEQ. ID No. 1 | AAEYKTSEVIGLGYSNGANIMANLLIEKGRVEDKAALLHPLVPFRP |
| | KDNPALEGAKILMTAGRMDPICPPDLTEALAQYFERQKADVELV |
| | WHPGGHELRQTELAAVQSLLAY |

### Compositions

A method for treating an animal who has been exposed to pyrethroid comprises administering an effective amount of a pyrethroid detoxifying enzyme to prevent or treat pyrethroid toxicity. The pyrethroid detoxifying enzyme is one or more hydrolyzing carboxylesterases.

Animals may include humans, aquatic species, a poultry species, a porcine species, a bovine species, an ovine species, an equine species, and companion animals, such as canines and felines.

The pyrethroid detoxifying enzyme may be administered by enteric/enteral route, parenteral or topical route.

The pyrethroid detoxifying enzymes of the present invention may be incorporated into pharmaceutical compositions that may be delivered to a subject, so as to allow delivery of a biologically active enzyme. In a particular embodiment of the present invention, pharmaceutical compositions comprising sufficient genetic material to enable a recipient to produce a therapeutically effective amount of a pyrethroid detoxification. An effective amount of the enzyme may be directly injected or infused into a patient in need thereof. The compositions may be administered alone or in combination with at least one other agent, such as a stabilizing compound, which may be administered in any sterile, biocompatible pharmaceutical carrier, including, but not limited to, saline, buffered saline, dextrose, and water. The compositions may be administered to a patient alone, or in combination with other agents.

The pharmaceutical compositions may also contain a pharmaceutically acceptable excipient. Such excipients include any pharmaceutical agent which may be administered without undue toxicity. Pharmaceutically acceptable excipients include, but are not limited to, liquids such as water, saline, glycerol, sugars and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles.

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

The pharmaceutical composition may be provided as a salt and can be formed with many acids, including but not limited to, hydrochloric, sulfuric, acetic, lactic, tartaric, malic, succinic, etc.

The pharmaceutical preparation may contain dosages of between IV push or injection 0.001 mg-1 g/kg/dose of pytH2 for IV push or injection. An oral dose pharmaceutical preparation may contain 1 mg-5 g/kg pytH2 per dose.

In one embodiment, the enzyme is administered to an animal orally or through feeding. The enzyme is administered to the animal a feed composition or drinking water comprising an effective amount of an additive one or more hydrolyzing carboxylesterases wherein the one or more hydrolyzing carboxylesterases causes an effect selected from the group consisting of preventing or treating pyrethroid toxicity.

In one embodiment, the enzyme administered to an animal comprises or consists of the amino acid sequence of SEQ ID NO: 1, 2, 3, 4 or 5, an allelic variant thereof; or is a fragment thereof able to degrade one or more pyrethroids.

The isolated enzyme can comprise an amino acid sequence having at least 75% identity to SEQ. ID Nos. 1, 2, 3, 4, or 5.

The isolated enzyme can comprise an amino acid sequence having at least 80% identity to SEQ. ID Nos. 1, 2, 3, 4, or 5.

The isolated enzyme can comprise an amino acid sequence having at least 85% identity to SEQ. ID Nos. 1, 2, 3, 4, or 5.

The isolated enzyme can comprise an amino acid sequence having at least 90% identity to SEQ. ID Nos. 1, 2, 3, 4, or 5.

The isolated enzyme can comprise an amino acid sequence having at least 95% identity to SEQ. ID Nos. 1, 2, 3, 4, or 5.

The isolated enzyme can comprise an amino acid sequence having at least 98% identity to SEQ. ID Nos. 1, 2, 3, 4, or 5.

The isolated enzyme can comprise an amino acid sequence having at least 99% identity to SEQ. ID Nos. 1, 2, 3, 4, or 5.

Enzymes can be prepared by a number of standard biochemical and molecular biology methods which are generally known in the art. For example, a gene encoding an enzyme can be amplified from chromosomal DNA using the polymerase chain reaction (PCR), and cloned into a suitable vector (e.g., a plasmid vector). The vector suitably comprises a multiple cloning site into which the DNA molecule encoding the fusion protein can be easily inserted. The vector also suitably contains a selectable marker, such as an antibiotic resistance gene, such that microorganisms transformed, transfected, or mated with the vector can be readily identified and isolated. Where the vector is a plasmid, the plasmid suitably also comprises an origin of replication. Alternatively, DNA coding for the enzyme protein can be integrated into the chromosomal DNA of the microorganism host.

The host can then be cultured and enzyme harvested from the cultures. A crude cell extract can be used or the enzyme can be partially or substantially purified using standard biochemical techniques.

Suitable hosts for large-scale production of enzymes include but are not limited to Bacillus species (e.g., Bacillus subtilis, Bacillus licheniformis, Bacillus coagulans, Bacillus megaterium, Bacillus thuringiensis, Bacillus fusiformis, Bacillus cereus, or Bacillus mycoides), Escherichia coli, Aspergillus niger, Aspergillus oryzae, Streptomyces species, Klebsiella species, Mucor species, Rhizopus species, Mortierella species, Kluyveromyces species, Candida species, Penicillium chrysogenum, Trichoderma species Saccharomyces cerevisiae, Pichia pastoris, Hansenula polymorpha, Kluyveromyces lactis, Yarrowia lipolytica, Schizosaccharomyces pombe, and Candida utilitis.

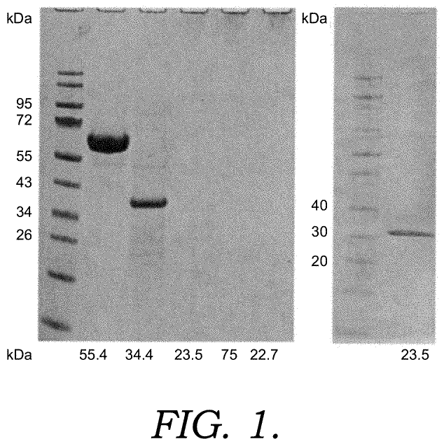

Enzymes can be expressed with a tag (e.g., a polyhistidine (His)) to facilitate purification as can be seen in FIG. 1.

pNBA Method

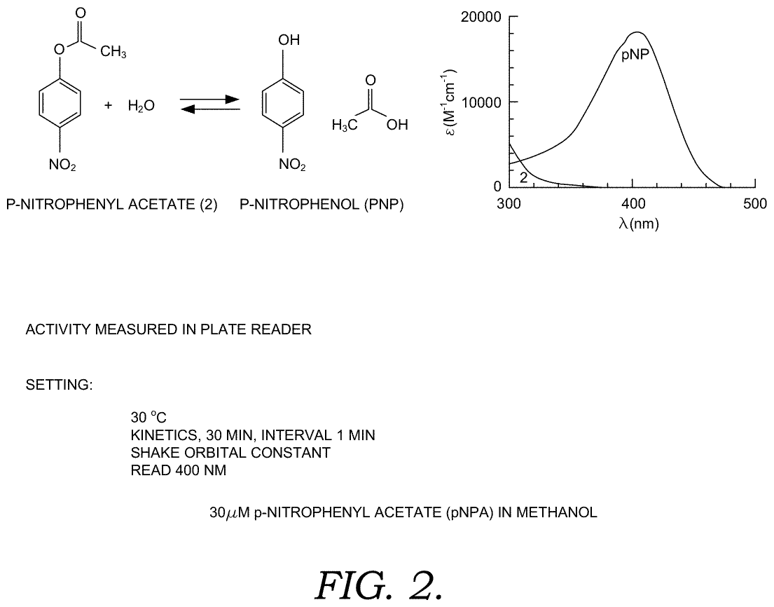

Each of the identified enzymes were evaluated for esterase activity assay using pNBA method described below. Determination of carboxylesterase activity of cargo enzyme in Bacillus thuringiensis spore productions or of free enzyme productions. Three active carboxylesterases cloned into pBCm (Bt EO) and pET28a (E. coli BL21) systems: pnbA (B. subtilis), PytH2 (Sphingobium wenxiniae strain), and MsE1 (Methylobacterium sp). p-Nitrophenyl acetate was the substrate in assays for esterase activity. The hydrolysis of p-nitrophenyl acetate releases p-nitrophenol and acetate, and while p-nitrophenyl acetate is colorless, p-nitrophenol is yellow. This allows to follow the reaction progress by measuring the generation of yellow color, the absorbance may be monitored between 400 and 410 nm where it shows the max absorbance in basic conditions as shown in FIG. 2.

6 mg p-Nitrophenyl acetate (pNPA, CAS #830-03-5, MW: 181.1 g/mol) was weighed and dissolved in 1 mL methanol (CAS #67-56-1, MW: 32.04 g/mol). Stored at −20° C. and kept on ice when preparing reactions.

Phosphate Buffer Solution (PBS)

8.0 g sodium chloride (NaCl, CAS #7647-14-5, MW: 58.44 g/mol), 1.44 g sodium phosphate dibasic was dissolved (Na2HPO4, CAS #7558-79-4, MW: 141.96 g/mol), and 0.24 g potassium phosphate monobasic (KH2PO4, CAS #7778-77-0, 136.09 g/mol) in 800 mL DI H2O, then adjust pH to 7.4 using hydrochloric acid (HCl, CAS #7647-01-0, MW: 36.46 g/mol). Make 100 mL aliquots and autoclave.

1 mg/mL Esterase from Rabbit Liver in PBS

Esterase from rabbit liver (CAS #9016-18-6, Sigma E0887-powder). Store at −20° C. To prepare a stock dissolve 1 mg in 1 mL PBS, kept on ice when using.

Up to 1 mL spore production was pelleted up for 5 min at max speed. Supernatant was discarded without disturbing spore pellet and suspend pellet in ice-cold PBS. Spores kept suspended on ice until use.

The assay was run at 30° C. for 30 minutes. The assay was shaken continuously. Activity was read on (A) 400 BioTek Synergy/HTX microplate reader was used. The results of pNPA hydrolysis are shown in Table 3 depicting carboxylesterase enzyme activity for each of the five strains using pNPA hydrolysis.

TABLE 3

| | | | pNPA hydrolysis |
|---|---|---|---|
| | | | [nmol pNPA/ |
| Abbreviation | Enzyme | Source | min/1E+8 spores |
| E1 | pytZ | Ochrobactrum sp. | BDL |
| E2 | pnbA | B. subtilis | 19.7 ± 1.4 |
| E3 | PytH2 | Sphingobium | 26.3 ± 0.9 |
| | | wenxiniae | |
| E4 | MsE1 | Methylobacterium sp. | 30.6 ± 0.0 |
| E5 | EstP | Klebsiella sp. | BDL |

BDL—below the detectable limit of the assay

### Permethrin Hydrolysis

The three enzymes, pnbA, pytH2 and msE1 and control were identified, expressed and tested in the quantifiable esterase assay.

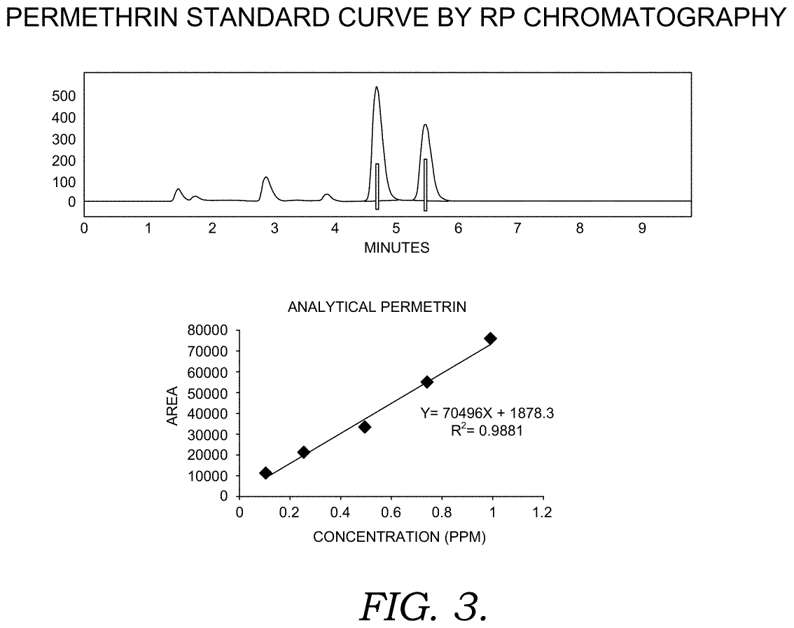

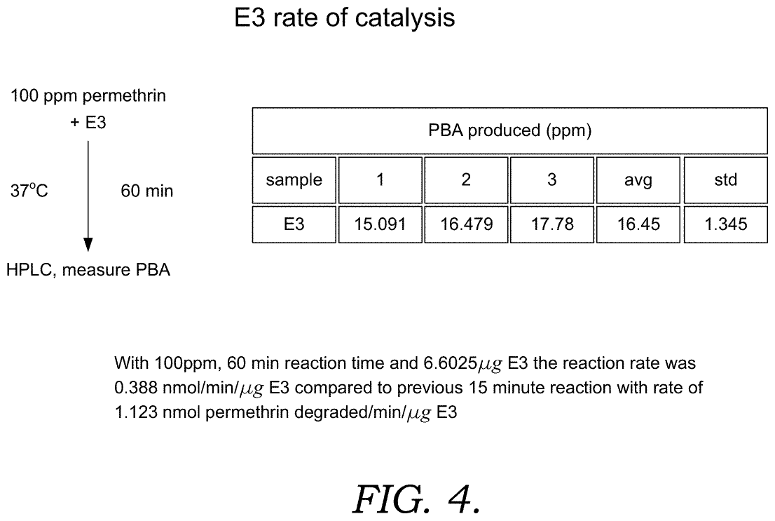

Permethrin hydrolysis was then performed for control as shown in FIGS. 3 and 4, and three esterases pnbA, pytH2 and MsE1. 3 μL of 83,333 ppm permethrin was added to 497 μL MeOH to make 500 μL of 500 ppm permethrin. 10 μL of 500 ppm permethrin was added to 940 μL PBS. 50 μL 2.63 mg/mL pPytH2PytH2 was added to the PBS/permethrin mixture to make a 5 ppm permethrin, 132 μg/mL pytH2 enzymatic reaction mixture. The enzymatic mixture was then incubated at 37 C for 1 hour. Samples of the enzymatic reaction were taken before and after the reaction (T0 before enzymatic reaction and T1) 1 hour after reaction.

400 μL of the enzymatic reaction was added to 600 uL MeOH to make a 2 ppm permethrin solution in an HPLC sample vial. A volume of each sample was injected into Waters 2695 Separations Module with a 2996 Photodiode Array Detector. Samples were run isocratically (85% acetonitrile and 15% water). Analysis was carried out at 254 nm and compared to a 5-point 2-fold standard curve for permethrin (Trans-permethrin and Cis-permethrin). The treatments from the permethrin hydrolysis are fully described in Table 4 below. They are reported as absolute remaining from the initial starting concentration and the percent permethrin concentration relative to the T0 control treatment.

TABLE 4
Permethrin hydrolysis by esterase proteins expressed in E0

Enzymatic reaction
1 mL reactions (PBS:MeOH)
20 ppm (20 ug/mL)
2.44 product
Incubation
1 h

| Esterase | |
|---|---|
| (2.44 1E+8 spores) | % Permethrin hydrolysis |
| BcIA₂₀₋₃₅−E2 | 7.50 |
| BcIA₂₀₋₃₅−E3 | 9.18 |
| BcIA₂₀₋₃₅−E4 | 4.75 |

### Cypermethrin Degradation

50 ppm of cypermethrin was placed in 1 mL PBS with 10 μL pPytH2, PytH2 and reacted for 15 minutes at 37 C with shaking at 300 rpm. HPLC was run on the sample after being diluted 1:1 MeOH using instrument method described above for permethrin for 10 minutes. Cypermethrin was degraded at a rate of 12.69 nmol/min/μg pPytH2,PytH2.

### Deltamethrin Degradation

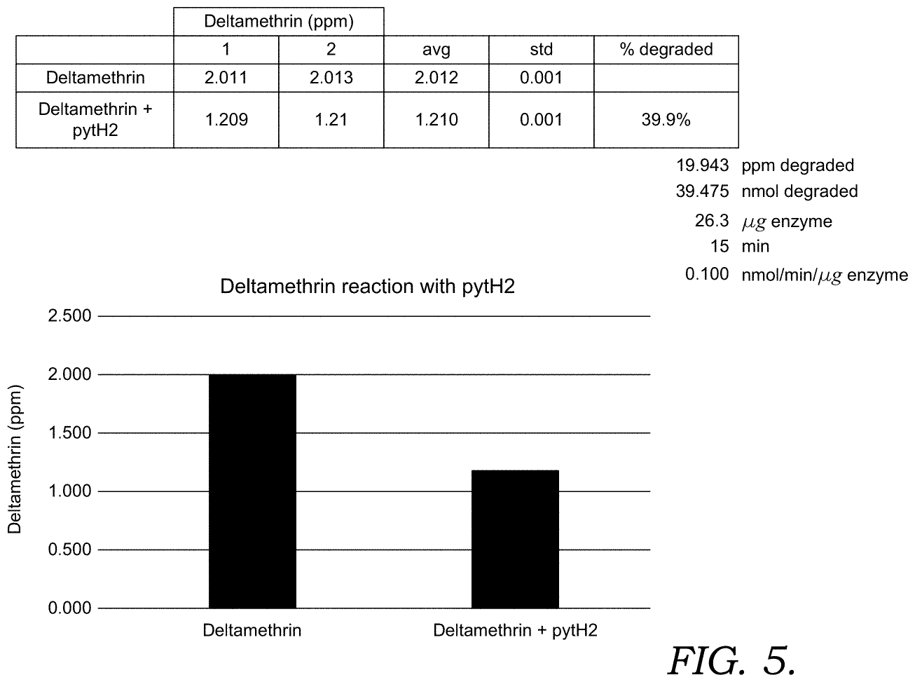

Using the method and instruments described above for permethrin, 40% of deltamethrin was degraded by pPytH2, PytH2 esterase as shown in FIG. 5.

### Fenpropathrin Degradation

1000 ppm fenpropathrin was diluted to 5 point curve which was able to detect on HPLC the gradient of 20% water and 80% using the instrument method described above for permethrin. 20 ppm of fenpropathrin was placed in 1 uL PBS with 10 μL fenpropathrin and reacted for 15 minutes at 37 C with shaking. HPLC was run on the sample after being diluted 1:1 MeOH using instrument method described above for permethrin. A 90% degradation of fenpropathrin was detected with PytH2.

### Various Matrices and Permethrin

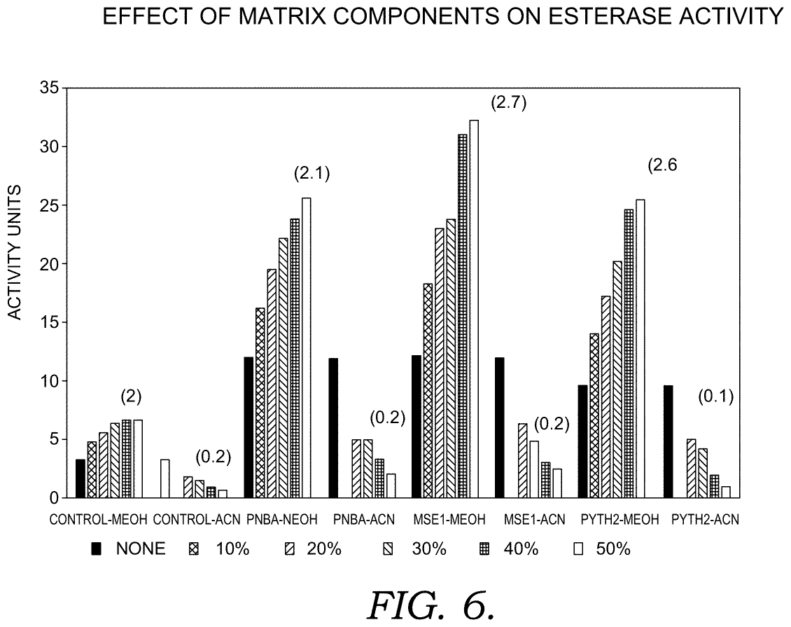

An assay was developed to determine the effect of matrix components on esterase activity with identified enzymes, pnbA, pytH2 and msE1 and control were identified, expressed and tested with various matrices. The results are shown in FIG. 6.

### Biological Permethrin Assay

A biological permethrin assay was performed for control, and three esterases pnbA, pytH2 and MsE1. The sensitivity of scuds (Hyalella azteca) to permethrin was tested with three esterases pnbA, pytH2 and MsE1.

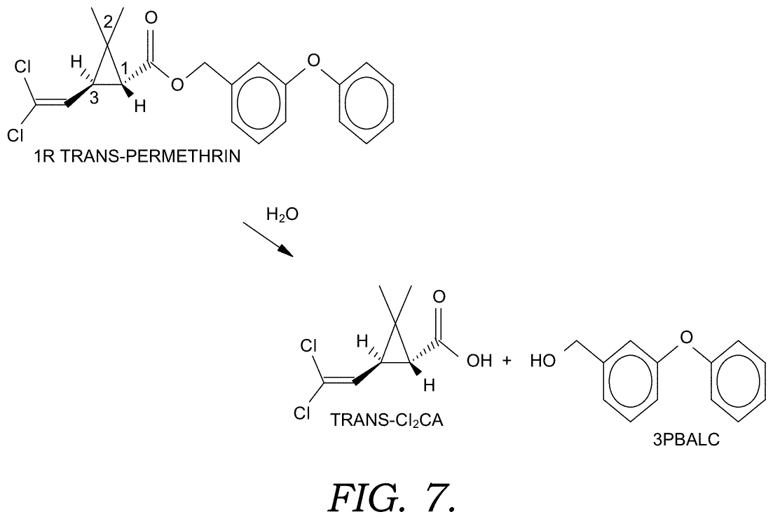

The LD50 of the crickets by permethrin was determined and a delivery mechanism was created. Each of the three esterases pnbA, pytH2 and MsE1 were tested to determine their ability to prevent lethality to the insects as seen in FIG. 7.

Analytical Demonstration of permethrin degradation

| Bioassay methods | Analytical methods |
|---|---|
| Cricket killing | Reverse Phase Chromotography |
| Drosophila killing | |
| Scud killing (preferred) | Spectroscopic analysis |

Insecticides have broad-spectrum toxicities and are lethal to multiple non target aquatic invertebrates in contaminated areas. Standard methods have been developed for assessing the toxicity of contaminants associated with sediments using amphipods and other invertebrates. The amphipod Hyalella azteca (scuds) is broadly used to assess such toxicities because scuds are relatively sensitive to chemicals, have contact with sediment, are easy of culture in laboratory, and are tolerant to varying culture conditions.

Two fish tanks with clean aquarium gravel were prepared and half of scuds were transferred to each tank. Two volumes of scud water were added to each tank. The culture was aerated and a bubbler was introduced and turned on in each tank. Scuds were fed every other week by adding ¼ full spoon of fish food.

Scuds that are close to 3 mm in length were selected and transferred to each container. Ten scuds were placed back in large glass pan with 3 mm² grid underneath. Each scud was individually selected and placed in a 150 mL beaker that is filled with 50 mL scud water. A separate beaker was used for one scud for one treatment.

For control and each of the three esterases pnbA, pytH2 and MsE1, ten scuds were exposed to permethrin and one of pnbA, pytH2 or MsE1.

Biological permethrin assay for esterase activity

| Esterases: | Activity units |
|---|---|
| Control | 8.1 |
| pnbA | 31.4 |
| pytH2 | 31.6 |
| MsE1 | 39.5 |

### Reaction Mix:

100 μL 50% MeOH in PBS

0.2 μg/μL

### Incubation:

37 degree C.

18 hr

Shanking 1000 rpm

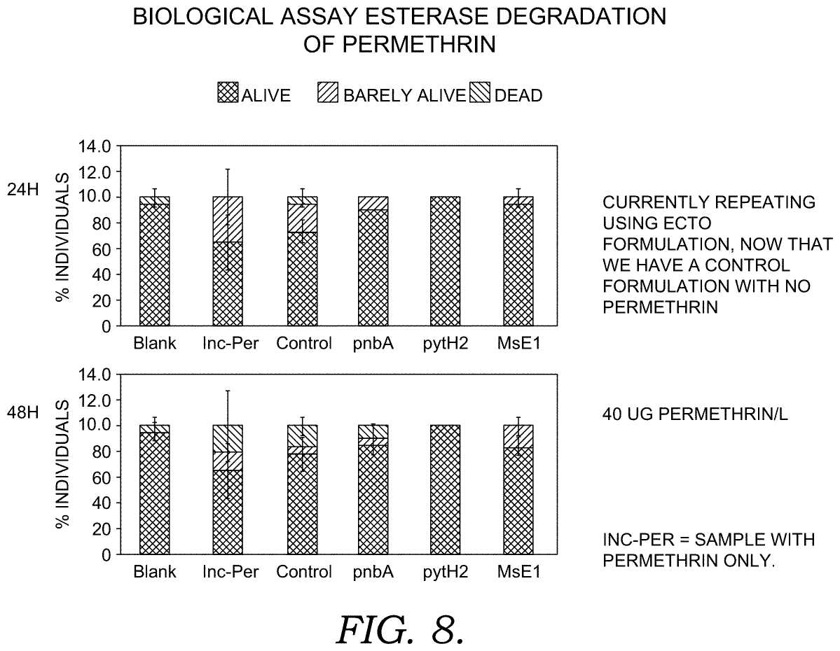

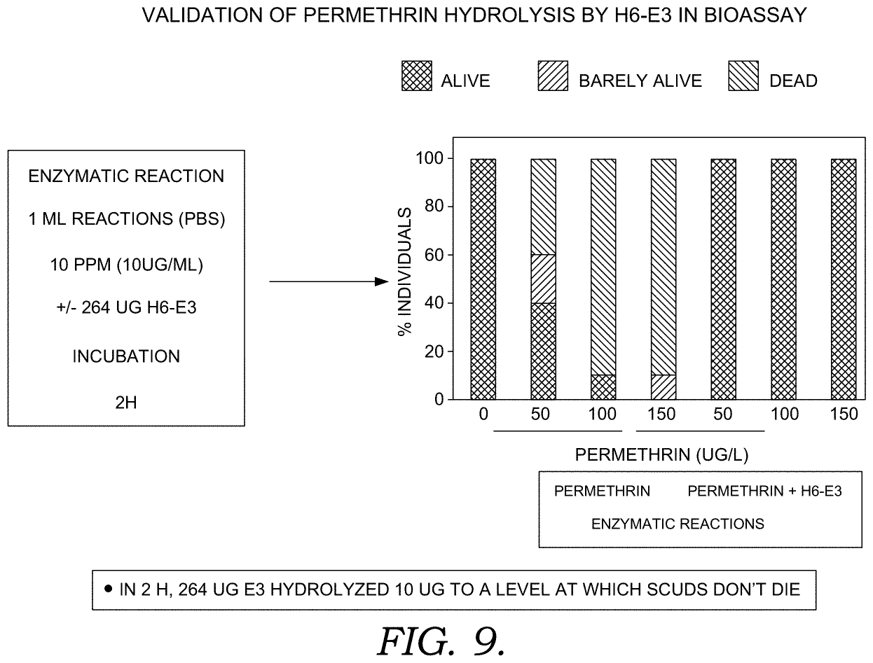

Transferred 20 μL rxn mix [4 μg] to 100 mL [containing 10 scuds]
At time, 24 h and 48 h the ten scuds were scored:
A=Alive and Moving actively
BA=Barely Alive—Difficulty swimming away; only legs are moving
D=Dead—Not moving at all
The results of which are shown in FIGS. 8 and 9.
Producing pPytH2PytH2 Carboxylesterase

The esterase gene (PytH2) of Sphingobium wenxiniae was synthesized as a DNA fragment containing an open reading frame (ORF) of 873 bp. The ORF of PytH2 was isolated by PCR and incorporated into the pET28a plasmid by homologous recombination. This plasmid was transformed into E. coli BL21 (DE3) and verified by sequencing. The esterase was expressed in Escherichia coli, under the control of T7 system. The expression plasmid, pET28a, used to introduce the PytH2 gene in the recipient strain is based on the replication origin of E. coli, on pBR322 and is selected for with the bacterial resistance gene KanR. The plasmid contains the expression cassette consisting of a T7Iac promoter, a fragment of the Sphingobium wenxiniae PytH2 (Pyrethroid hydrolase) gene encoding the esterase activity, N-terminal His tag, and the transcriptional terminator of T7.

Protein is a carboxylesterase from Sphingobium wenxiniae produced by PytH2 recombinant, expressed in E. coli BL21 (DE3) IUBMB: 3.1.1.88 CAS: 9016-18-6. The Chemical Composition of the carboxylesterase has Chemical purity: >90% buffer of 10 mM phosphate (pH 7.4), 2.7 mM KCl, 137 mM NaCl. Post translational procedures produced in a protease deficient E. coli strain and optimized for synthesis. The protein Appearance: clear, colorless solution:

### Physical State: Liquid

pH: 7.4
Water Solubility: Not applicable

### Storage: −20° C.

Unit definition: One unit is defined as the amount of enzyme required to generate 1 μmol/min of 4-p-nitrophenol from 4-nitrophenyl-L-acetate at 30° C. and pH 7.4.
Specific activity: 15±3 μ/mg

This enzyme is quite stable, and does not require a cofactor for its activity. It becomes inactive after incubation at 70° C. The enzyme is strongly inhibited by metal ions, but can recover activity. The enzyme is also deactivated by surfactant compounds such as SDS. Activity of the enzyme: In order of hydrolysis efficiency: transpermethrin>cis-permethrin>fenpropathrin>trans-cypermethrin>cis-cypermethrin>cyhalothrin>fenvalerate>deltamethrin>bifenthrin.

An esterase gene (PytH2) of Sphingobium wenxiniae containing an open reading frame (ORF) of 873 bp. The ORF of PytH2 was expressed in Escherichia coli, under the control of the promoter T7. The amino acid sequence of PytH2 indicated that the esterase is a novel member of the A/B hydrolase family of enzymes and that the enzyme contains a catalytic triad, consisting of Ser78, Asp202, and His230 and a structural motif, GHSLG tight turn. E. coli BL21(DE3)/pET28 containing PytH2 expressed a novel 31.4-kDa protein corresponding to PytH2 in an N-terminal fusion with the His-tag peptide. The recombinant Sphingobium wenxiniae PytH2 protein was purified to electrophoretic homogeneity in a one-step affinity chromatography procedure on Ni-NTA Resin. The optimum pH and temperature of the purified enzyme were 7.0 and 37 degrees C., respectively. Among the pNP (p-nitrophenyl) esters tested, pNP-acetate (C(2)), was the best substrate. It was also active on pNP-butyrate(C(4) and pNPcaproate(C(6)).

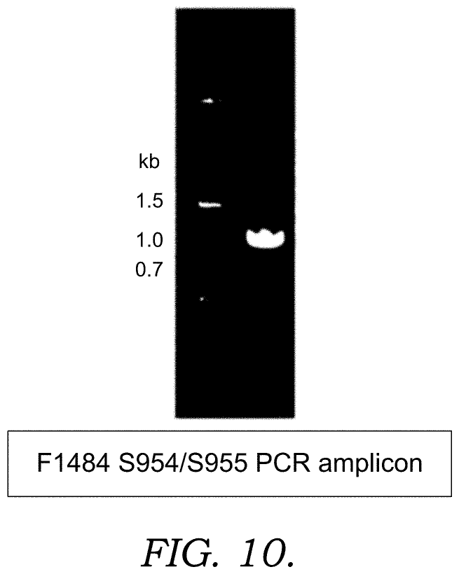

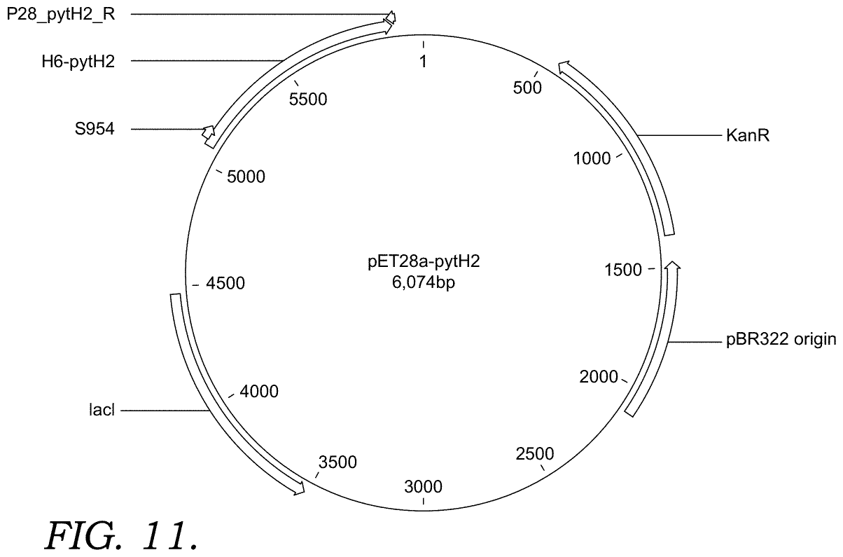

PytH2 SEQ. ID No. 6 840 base pairs
5′-ATG ACT GTA ACC GAT ATC ATA CTC ATT CAC GGA GCG CTC AAC CGT GGT GCC TGT TAC GAT GCG GTC
GTC CCG CTG TTA GAG GCC CGC GGA TAT CGT GTC CAT GCT CCT GAC CTG ACA GGG CAC ACA CCA GGC
GAC GGG GGA CAT CTT AGT GTA GTA GAC ATG GAA CAC TAT ACT CGC CCA GTA GCA GAC ATA CTC GCT
CGG GCT GAA GGC CAA TCC ATC CTG TTA GGA CAC AGC CTG GGC GGC GCC TCT ATC TCT TGG TTG GCC
CAA CAC CAC CCA GAC AAA GTT GCT GGT CTC ATT TAT CTG ACC GCC GTT TTG ACA GCG CCA GGC ATA
ACA CCG GAG ACA TTC GTT TTA CCA GGC GAG CCA AAT CGT GGA ACG CCT CAT GCA CTC GAC TTG ATC
CAA CCA GTT GAT GAG GGA CGG GGA TTG CAG GCT GAT TIC TCC CGG TTA GAG AGA CTT CGT GAG GTC
TTT ATG GGC GAC TAC CCT GGC GAA GGG ATG CCG CCT GCA GAG CAG TTT ATT CAA ACA CAG TCA ACA
GTG CCG TTT GGT ACG CCT AAT CCA ATG GAG GGG CGT GCT CTT GAA ATT CCT CGC CTT TAT ATA GAA
GCG CTT GAC GAT GTG GTC ATA CCG ATA GCC GTC CAA CGC CAA ATG CAA AAG GAG TTT CCA GGC CCG
GTG GCT GTC GTA TCA CTC CCA GCT AGC CAC GCA CCT TAG TAT AGT ATG CCA GAG AGA CTT GCG GAG
GCA ATT GCC GAT TTT GCA GAT GCC CCG GCC GAA TAT CGT CAG ACT GCC ACA AAG GCG GGC CCT GAT
AGA CCG GCA GGG GCG GAC GGT GGC CGG GCT GAC CGT GCG GAT CTG CCA-3′
PytH2 SEQ. ID No. 5 280 as
MTVTDIILIHGALNRGACYDAVVPLLEARGYRWAPDLTGHTPGDGGHL5VVDMEWYTRPVADILARAEGQSILLGESL
GGASISWLAQHHPDKVAGLIYLTAVLTAPGITPETFVLFGEPNRGTPHALDLIQPVDEGRGLQADFSRLERLREVFMGDY
PGEGMPPAEQFIQTQSTVPFGTPNPMEGRALEIPRLYIEALDDVVIPIAVQRQMQKEETGPVAVVSL2ASHAPYYSMPER
LAEAIADFADAPAEYRQTATKAGPDRPAGADGGRADRADLP
Primers below used for verification of PytH2 in plasmid via PCR:
(S954) CATCATCACAGCAGCGGAATGACTGTAACCGATATCATACTCATTCACGGAGC
(S955) TTTCGGGCTTTGTTATGGCAGATCCGCACGGTCA
Expected amplicon size:
(pET28a-PytH2) 0.985 kb as seen in FIG. 10.
Nucleotide sequence plasmid SEQ. ID No. 7 as seen in FIG. 11
Name-pET28a-PytH2
TGGCGAATGGGACGCGCCCTGTAGCGGCGCATTAAGCGCGGCGGGTGTGGTGGTTACGCGCAGCGTGACCGCTACACTTGCCAGCGCC
CTAGCGCCCGCTCCTTTCGCTTTCTTCCCTTCCTTTCTCGCCACGTTCGCCGGCTTTCCCCGTCAAGCTCTAAATCGGGGGCTCCCTT
TAGGGTTCCGATTTAGTGCTTTACGGCACCTCGACCCCAAAAAACTTGATTAGGGTGATGGTTCACGTAGTGGGCCATCGCCCTGATA
GACGGTTTTTCGCCCTTTGACGTTGGAGTCCACGTTCTTTAATAGTGGACTCTTGTTCCAAACTGGAACAACACTCAACCCTATCTCG
GTCTATTCTTTTGATTTATAAGGGATTTTGCCGATTTCGGCCTATTGGTTAAAAAATGAGCTGATTTAACAAAAATTTAACGCGAATT
TTAACAAAATATTAACGTTTACAATTTCAGGTGGCACTTTTCGGGGAAATGTGCGCGGAACCCCTATTTGTTTATTTTTCTAAATACA
TTCAAATATGTATCCGCTCATGAATTAATTCTTAGAAAAACTCATCGAGCATCAAATGAAACTGCAATTTATTCATATCAGGATTATC
AATACCATATTTTTGAAAAAGCCGTTTCTGTAATGAAGGAGAAAACTCACCGAGGCAGTTCCATAGGATGGCAAGATCCTGGTATCGG
TCTGCGATTCCGACTCGTCCAACATCAATACAACCTATTAATTTCCCCTCGTCAAAAATAAGGTTATCAAGTGAGAAATCACCATGAG
TGACGACTGAATCCGGTGAGAATGGCAAAAGTTTATGCATTTCTTTCCAGACTTGTTCAACAGGCCAGCCATTACGCTCGTCATCAAA
ATCACTCGCATCAACCAAACCGTTATTCATTCGTGATTGCGCCTGAGCGAGACGAAATACGCGATCGCTGTTAAAGGACAATTACAAA
CAGGAATCGAATGCAACCGGCGCAGGAACACTGCCAGCGCATCAACAATATTTTCACCTGAATCAGGATATTCTTCTAATACCTGGAA
TGCTGTTTTCCCGGGGATCGCAGTGGTGAGTAACCATGCATCATCAGGAGTACGGATAAAATGCTTGATGGTCGGAAGAGGCATAAAT
TCCGTCAGCCAGTTTAGTCTGACCATCTCATCTGTAACATCATTGGCAACGCTACCTTTGCCATGTTTCAGAAACAACTCTGGCGCAT
CGGGCTTCCCATACAATCGATAGATTGTCGCACCTGATTGCCCGACATTATCGCGAGCCCATTTATACCCATATAAATCAGCATCCAT
GTTGGAATTTAATCGCGGCCTAGAGCAAGACGTTTCCCGTTGAATATGGCTCATAACACCCCTTGTATTACTGTTTATGTAAGCAGAC
AGTTTTATTGTTCATGACCAAAATCCCTTAACGTGAGTTTTCGTTCCACTGAGCGTCAGACCCCGTAGAAAAGATCAAAGGATCTTCT
TGAGATCCTTTTTTTCTGCGCGTAATCTGCTGCTTGCAAACAAAAAAACCACCGCTACCAGCGGTGGTTTGTTTGCCGGATCAAGAGC
TACCAACTCTTTTTCCGAAGGTAACTGGCTTCAGCAGAGCGCAGATACCAAATACTGTCCTTCTAGTGTAGCCGTAGTTAGGCCACCA
CTTCAAGAACTCTGTAGCACCGCCTACATACCTCGCTCTGCTAATCCTGTTACCAGTGGCTGCTGCCAGTGGCGATAAGTCGTGTCTT
ACCGGGTTGGACTCAAGACGATAGTTACCGGATAAGGCGCAGCGGTCGGGCTGAAEGGGGGGTTCGTGCACACAGCCCAGCTTGGAGC
GAACGACCTACACCGAACTGAGATACCTACAGCGTGAGCTATGAGAAAGCGCCACGCTTCCCGAAGGGAGAAAGGCGGACAGGTATCC
GGTAAGCGGCAGGGTCGGAACAGGAGAGCGCACGAGGGAGCTTCCAGGGGGAAACGCCTGGTATCTTTATAGTCCTGTCGGGTTTCGC
CACCTCTGACTTGAGCGTCGATTTTTGTGATGCTCGTCAGGGGGGCGGAGCCTATGGAAAAACGCCAGCAACGCGGCCTTTTTACGGT
TCCTGGCCTTTTGCTGGCCTTTTGCTCACATGTTCTTTCCTGCGTTATCCCCTGATTCTGTGGATAACCGTATTACCGCCTTTGAGTG
AGCTGATACCGCTCGCCGCAGCCGAACGACCGAGCGCAGCGAGTCAGTGAGCGAGGAAGCGGAAGAGCGCCTGATGCGGTATTTTCTO
CTTACGCATCTGTGCGGTATTTCACACCGCATATATGGTGCACTCTCAGTACAATCTGCTCTGATGCCGCATAGTTAAGCCAGTATAC
ACTCCGCTATCGCTACGTGACTGGGTCATGGCTGCGCCCCGACACCCGCCAACACCCGCTGACGCGCCCTGACGGGCTTGTCTGCTCC
CGGCATCCGCTTACAGACAAGCTGTGACCGTCTCCGGGAGCTGCATGTGTCAGAGGTTTTCACCGTCATCACCGAAACGCGCGAGGCA
GCTGCGGTAAAGCTCATCAGCGTGGTCGTGAAGCGATTCACAGATGTCTGCCTGTTCATCCGCGTCCAGCTCGTTGAGTTTCTCCAGA
AGCGTTAATGTCTGGCTTCTGATAAAGCGGGCCATGTTAAGGGCGGTTTTTTCCTGTTTGGTCACTGATGCCTCCGTGTAAGGGGGAT
TTCTGTTCATGGGGGTAATGATACCGATGAAACGAGAGAGGATGCTCACGATACGGGTTACTGATGATGAACATGCCCGGTTACTGGA
ACGTTGTGAGGGTAAACAACTGGCGGTATGGATGCGGCGGGACCAGAGAAAAATCACTCAGGGTCAATGCCAGCGCTTCGTTAATACA
GATGTAGGTGTTCCACAGGGTAGCCAGCAGCATCCTGCGATGCAGATCCGGAACATAATGGTGCAGGGCGCTGACTTCCGCGTTTCCA
GACTTTACGAAACACGGAAACCGAAGACCATTCATGTTGTTGCTCAGGTCGCAGACGTTTTGCAGCAGCAGTCGCTTCACGTTCGCTC
GCGTATCGGTGATTCATTCTGCTAACCAGTAAGGCAACCCCGCCAGCCTAGCCGGGTCOTCAACGACAGGAGCACGATCATGCGCACC
CGTGGGGCCGCCATGCCGGCGATAATGGCCTGCTTCTCGCCGAAACGTTTGGTGGCGGGACCAGTGACGAAGGCTTGAGCGAGGGCGT
GCAAGATTCCGAATACCGCAAGCGACAGGCCGATCATCGTCGCGCTCCAGCGAAAGCGGTCCTCGCCGAAAATGACCCAGAGCGCTGC
CGGCACCTGTCCTACGAGTTGCATGATAAAGAAGACAGTCATAAGTGCGGCGACGATAGTCATGCCCCGCGCCCACCGGAAGGAGCTG
ACTGGGTTGAAGGCTCTCAAGGGCATCGGTCGAGATCCCGGTGCCTAATGAGTGAGCTAACTTACATTAATTGCGTTGCGCTCACTGC
CCGCTTTCCAGTCGGGAAACCTGTCGTGCCAGCTGCATTAATGAATCGGCCAACGCGCGGGGAGAGGCGGTTTGCGTATTGGGCGCCA
GGGTGGTTTTTCTTTTCACCAGTGAGACGGGCAACAGCTGATTGCCCTTCACCGCCTGGCCCTGAGAGAGTTGCAGCAAGCGGTCCAC
GCTGGTTTGCCCCAGCAGGCGAAAATCCTGTTTGATGGTGGTTAACGGCGGGATATAACATGAGCTGTCTTCGGTATCGTCGTATCCC
ACTACCGAGATATCCGCACCAACGCGCAGCCCGGACTCGGTAATGGCGCGCATTGCGCCCAGCGCCATCTGATCGTTGGCAACCAGCA
TCGCAGTGGGAACGATGCCCTCATTCAGCATTTGCATGGTTTGTTGAAAACCGGACATGGCACTCCAGTCGCCTTCCCGTTCCGCTAT
CGGCTGAATTTGATTGCGAGTGAGATATTTATGCCAGCCAGCCAGACGCAGACGCGCCGAGACAGAACTTAATGGGCCCGCTAACAGC
GCGATTTGCTGGTGACCCAATGCGACCAGATGCTCCACGCCCAGTCGCGTACCGTCTTCATGGGAGAAAATAATACTGTTGATGGGTG
TCTGGTCAGAGACATCAAGAAATAACGCCGGAACATTAGTGCAGGCAGCTTCCACAGCAATGGCATCCTGGTCATCCAGCGGATAGTT
AATGATCAGCCCACTGACGCGTTGCGCGAGAAGATTGTGCACCGCCGCTTTACAGGCTTCGACGCCGCTTCGTTCTACCATCGACACC
ACCACGCTGGCACCCAGTTGATCGGCGCGAGATTTAATCGCCGCGACAATTTGCGACGGCGCGTGCAGGGCCAGACTGGAGGTGGCAA
CGCCAATCAGCAACGACTGTTTGCCCGCCAGTTGTTGTGCCACGCGGTTGGGAATGTAATTCAGCTCCGCCATCGCCGCTTCCACTTT
TTCCCGCGTTTTCGCAGAAACGTGGCTGGCCTGGTTCACCACGCGGGAAACGGTCTGATAAGAGACACCGGCATACTCTGCGACATCG
TATAACGTTACTGGTTTCACATTCACCACCCTGAATTGACTCTCTTCCGGGCGCTATCATGCCATACCGCGAAAGGTTTTGCGCCATT
CGATGGTGTCCGGGATCTCGACGCTCTCCCTTATGCGACTCCTGCATTAGGAAGCAGCCCAGTAGTAGGTTGAGGCCGTTGAGCACCG
CCGCCGCAAGGAATGGTGCATGCAAGGAGATGGCGCCCAACAGTCCCCCGGCCACGGGGCCTGCCACCATACCCACGCCGAAACAAGC
GCTCATGAGCCCGAAGTGGCGAGCCCGATCTTCCCCATCGGTGATGTCGGCGATATAGGCGCCAGCAACCGCACCTGTGGCGCCGGTG
ATGCCGGCCACGATGCGTCCGGCGTAGAGGATCGAGATCTCGATCCCGCGAAATTAATACGACTCACTATAGGGGAATTGTGAGCGGA
TAACAATTCCCCTCTAGAAATAATTTTGTTTAACTTTAAGAAGGAGATATACCATGGGCAGCAGCCATCATCATCATCATCACAGCAG
CggaATGACTGTAACCGATATCATACTCATTCACGGAGCGCTCAACCGTGGTGCCTGTTACGATGCGGTCGTCCCGCTGTTAGAGGCC
CGCGGATATCGTGTCCATGCTCCTGACCTGACAGGGCACACACCAGGCGACGGGGGACATCTTAGTGTAGTAGACATGGAACACTATA
CTCGCCCAGTAGCAGACATACTCGCTCGGGCTGAAGGCCAATCCATCCTuriAGGACACAGCCTGGGCGGCGCCTCTATCTCTTGGTT
GGCCCAACACCACCCAGACAAAGTTGCTGGTCTCATTTATCTGACCGCCGTTTTGACAGCGCCAGGCATAACACCGGAGACATTCGTT
TTACCAGGCGAGCCAAATCGTGGAACGCCTCATGCACTCGACTTGATCCAACCAGTTGATGAGGGACGGGGATTGCAGGCTGATTTCT
CCCGGTTAGAGAGACTTCGTGAGGTCTTTATGGGCGACTACCCTGGCGAAGGGATGCCGCCTGCAGAGCAGTTTATTCAAACACAGTC
AACAGTGCCGTTTGGTACGCCTAATCCAATGGAGGGGCGTGCTCTTGAAATTCCTCGCCTTTATATAGAAGCGCTTGACGATGTGGTC
ATACCGATAGCCGTCCAACGCCAAATGCAAAAGGAGTTTCCAGGCCCGGTGGCTGTCGTATCACTCCCAGCTAGCCACGCACCTTACT
ATAGTATGCCAGAGAGACTTGCGGAGGCAATTGCCGATTTTGCAGATGCCCCGGCCGAATATCGTCAGACTGCCACAAAGGCGGGCCC
TGATAGACCGGCAGGGGCGGACGGTGGCCGGGCTGACCGTGCGGATCTGCCATAACAAAGCCCGAAAGGAAGCTGAGTTGGCTGCTGC
CACCGCTGAGCAATAACTAGCATAACCCCTTGGGGCCTCTAAACGGGTCTTGAGGGGTTTMGCTGAAAGGAGGAACTATATCCGGAT

### Serum Analysis

Cat serum was acquired, permethrin was added to the serum and was assayed as a control by HPLC. After the cat serum control was analyzed three different concentrations of the enzyme were introduced to the cat serum control and then analyzed by HPLC. The results of the analysis show that 10 μl enzyme concentration added to the poisoned cat serum degraded all of the permethrin and the HPLC analyzed 0.0% permethrin in the control cat serum after the enzyme was applied. The results are shown in Table 5.

TABLE 5

Serum plus permethrin in HPLC
Cat serum
+permethrin
+/−E3
15 min ↓ 37 degree C.
Extraction
w/MeOH
+filtering
↓
HPLC

| | | | | Total | Total | % |
|---|---|---|---|---|---|---|
| | Trans- | Cis- | Total | permethrin | permethrin | permethrin |
| Sample | permethrin | permethrin | Std | (area) | (expected 2 ppm) | control |
| No enzyme | 16163.75 | 18519 | 13% | 34682.75 | 1.402 | |
| 30 μL E3 | 0 | 0 | 0 | 0 | 0.000 | 0.0% |
| 10 μL E3 | 0 | 0 | 0 | 0 | 0.000 | 0.0% |
| 2.5 μL E3 | 0 | 1624.5 | 24% | 1624.5 | 0.098 | 7.0% |

In the serum there appears to be either an inability to recover all of the permethrin or a native esterase that has a preference for the trans isomer

1. A method for treating an animal who has been exposed to pyrethroid comprising administering an effective amount of a pyrethroid detoxifying enzyme to prevent or treat pyrethroid toxicity.

2. The method of claim 1, wherein the pyrethroid detoxifying enzyme is one or more hydrolyzing carboxylesterases.

3. The method of claim 1 wherein the animal is selected from the group consisting of an aquatic species, a poultry species, a porcine species, a bovine species, an ovine species, an equine species, and a companion animal.

4. The method of claim 3 wherein the animal is a companion animal.

5. The method of claim 4 wherein the animal is a canine species or a feline species.

6. The method of claim 5, wherein the animal is a feline species.

7. The method of claim 1, wherein the pyrethroid is one or more of pyrethrin, tetramethrin, allethrin, phenothrin, barthrin, dimethrin, bioresmethrin, permethrin fenpropathrin, bifenthrin, cyfluthrin, beta-cylfuthrin, phenothrin, imiprothrin, flumethrin, momfluorothrin, bioallethirn, deltamethrin, cypermethrin, and tetramethrin.

8. A composition for the administering to an animal who has been exposed to pyrethroid, the composition comprising a pharmaceutical carrier suitable for administration in an animal and an effective amount of a hydrolyzing carboxylesterase.

9. The composition of claim 8, wherein the composition is administered to an animal is selected from the group consisting of an aquatic species, a poultry species, a porcine species, a bovine species, an ovine species, an equine species, and a companion animal.

10. The composition of claim 9, wherein the animal is a companion animal.

11. The composition of claim 10, wherein the animal is a feline species.

12. The composition of claim 8, wherein the animal has been exposed to is one or more of pyrethrin, tetramethrin, allethrin, phenothrin, barthrin, dimethrin, bioresmethrin, permethrin, fenpropathrin, bifenthrin, cyfluthrin, beta-cylfuthrin, phenothrin, imiprothrin, flumethrin, momfluorothrin, bioallethirn, deltamethrin, cypermethrin, and tetramethrin.

13. A method for treating an animal to prevent pyrethroid toxicity, the method comprising: administering an effective amount of a pyrethroid detoxifying enzyme to prevent pyrethroid toxicity.

14. The method of claim 13, further comprising: administering an effective amount of a one or more hydrolyzing carboxylesterases to an animal to prevent the toxic effect of synthetic pyrethroids.

15. The method of claim 14, wherein the administration of an effective amount of one or more hydrolyzing carboxylesterases is done before the animal is exposed to pyrethroids.

16. A method of claim 15, wherein administering an effective amount of one or more hydrolyzing carboxylesterases an animal to reduces the toxic effect of synthetic pyrethroids.

17. The method of claim 13, wherein the pyrethroid detoxifying enzyme is one or more hydrolyzing carboxylesterases.

18. The method of claim 13, comprising feeding an animal, the method comprising the step of administering to the animal a feed composition or drinking water comprising an effective amount of an additive one or more hydrolyzing carboxylesterases wherein the one or more hydrolyzing carboxylesterases causes an effect selected from the group consisting of preventing or treating pyrethroid toxicity.

19. The method of claim 13, comprising administering to the animal an effective amount of hydrolyzing carboxylesterease by injection.

20. The method of claim 13, comprising administering to the animal an effective amount of hydrolyzing carboxylesterease by topical administration.


SEQUENCE LISTING (7 sequences)

SEQ ID NO: 1 - protein, 201 aa, Ochrobactrum
MTTQTYEHRLKAGAKGAPLFIVFHGTGGDENQFFGLAEQLLPDATIMSPRGDVSEYGAARFFRRTGEGVYDMEDLARATDKMAGFIAALAAEYKTSEVIGLGYSNGANIMANLLIEKGRVEDKAALLHPLVPFRPKDNPALEGAKILMTAGRMDPICPPDLTEALAQYFERQKADVELVWHPGGHELRQTELAAVQSLLAY

SEQ ID NO: 2 - protein, 491 aa, Bacillus subtilis
MTHQIVTTQYGKVKGTTENGVHKWKGIPYAKPPVGQWRFKAPEPPEVWEDVLDATAYGSICPQPSDLLSLSYTELPRQSEDCLYVNVFAPDTPSKNLPVMVWIHGGAFYLGAGSEPLYDGSKLAAQGEVIVVTLNYRLGPFGFLHLSSFNEAYSDNLGLLDQAAALKWVRENISAFGGDPDNVTVFGESAGGMSIAALLAMPAAKGLFQICAIMESGASRTMTICEQAASTSAAFLQVLGINEGQLDKLHTVSAEDLLKAADQLRIAEKENFQLFFQPALDPKTLPEEPEKAIAEGAASGIPLLIGTTRDEGYLFFTPDSDVHSQETLDAALEYLLGKPLAEKVADLYPRSLESQIHMMTDLLEWRPAVAYASAQSHYAPVWMYREDWHPKKPPYNKAFHALELPFVFGNLDGLERMAKAEITDEVKQLSHTIQSAWITFAKTGNPSTEAVNWPAYHEETRETLILDSEITIENDPESEICRQKLEPSKGE

SEQ ID NO: 3 - protein, 279 aa, Sphingobium wenxiniae
MTVTDIILIHGALNRGACYDAVVPLLEARGYRVHAPDLTGHTPGDGGHLSVVDMEHYTRPVADILARAEGQSILLGHSLGGASISWLAQHHYDKVAGLIYLTAVLTAPGITPETFVLPGEPNRGTPHALDLIQPVDEGRGLQADFSRLERLREVFMGDYPGEGMPPAEQFIQTQSTVPFGTPNPMEGRALEIPRLYEALDDVVIPIAVQRQMQKEFPGPVAVVSLPASHAPYYSMPERLAEAIADFADAPAEYRQTATKAGPDRPAGADGGRADRADLP

SEQ ID NO: 4 - protein, 211 aa, Methylobacterium
MTQDTTGFIFIREEPGPDTTRPLMLLHGTGGDENDLLPLGRMVAPEAALLAPRGGVSENGMPRFFRRLAEGVFDEADLRRRTGDLAAFVAASRARYGLGAPLALGFSNGANIAASLLMLRPETLTGAVLIRPMVPFAEPPAADLAGRPVLILSGAMDPIVPVENARRLAQQLSASGARVEHRILPAGHGLSQADVSQALAWLRSLPGPEAA

SEQ ID NO: 5 - protein, 639 aa, Klebsiella
MEICTKGSRKHLTSRASEPSYNVPENQYVLYVVSSTLSIVICQLVKVAESKKSRFSGAIEKLNERLDSLKDYRIINRDLVVKDLERLKKRFDTEVINAELSEQLAKINVNLSRSYSEKGYLRLEKATGSENEWWAKKPSQNDDWQQFEPDGYTIFSSRDHYASLEKSYSDYEIEAKIPLPLRRGICAVVLYPEYISRICVLPESRSIQREQENFTKLRDKGIALSKKDWQAKLTIDELAEQEKERATINKRLGYFATEHEKVGIVHKALEPKLICPFQQIEKEWRQCKVKSKSTFPNSMTFVQNPAYQAVHSGFKKLKEQIGLADEDILLSLEKIEAIGLVNMPLLYERWCLLQIIKVLTQAFRYQPEDNWKRKLIANIQGNEEQISIQFFNPSVSRAITLQYEPFLANGKRPDFVLDVEAITKSGNQISKRLVVDAKYYSAAYLKQRGGIGGVIHELYNGKDYSECQENSVFVLHPVLDAVEKVVSPQEWAKDSYLGELSMFDWEPAHHQRQATNYGAVCANPMKSQRYLDEIQRMLGMFLQYGIEDNTSFRGASDDTHAVNFCVSCGSEKVVDVTKSMSSNNQKRWYRCNECTHFTVYTHCGTCNTRLIKNGEYWTYLSLMPMSSINIKCPNCESPV

SEQ ID NO: 6 - DNA, 840 bp, Sphingobium wenxiniae
atgactgtaaccgatatcatactcattcacggagcgctcaaccgtggtgcctgttacgatgcggtcgtcccgctgttagaggcccgcggatatcgtgtccatgctcctgacctgacagggcacacaccaggcgacgggggacatcttagtgtagtagacatggaacactatactcgcccagtagcagacatactcgctcgggctgaaggccaatccatcctgttaggacacagcctgggcggcgcctctatctcttggttggcccaacaccacccagacaaagttgctggtctcatttatctgaccgccgttttgacagcgccaggcataacaccggagacattcgttttaccaggcgagccaaatcgtggaacgcctcatgcactcgacttgatccaaccagttgatgagggacggggattgcaggctgatttctcccggttagagagacttcgtgaggtctttatgggcgactaccctggcgaagggatgccgcctgcagagcagtttattcaaacacagtcaacagtgccgtttggtacgcctaatccaatggaggggcgtgctcttgaaattcctcgcctttatatagaagcgcttgacgatgtggtcataccgatagccgtccaacgccaaatgcaaaaggagtttccaggcccggtggctgtcgtatcactcccagctagccacgcaccttactatagtatgccagagagacttgcggaggcaattgccgattttgcagatgccccggccgaatatcgtcagactgccacaaaggcgggccctgatagaccggcaggggcggacggtggccgggctgaccgtgcggatctgcca

SEQ ID NO: 7 - DNA, 6074 bp, Artificial Sequence
  Synthetic
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcagcagccatcatcatcatcatcacagcagcggaatgactgtaaccgatatcatactcattcacggagcgctcaaccgtggtgcctgttacgatgcggtcgtcccgctgttagaggcccgcggatatcgtgtccatgctcctgacctgacagggcacacaccaggcgacgggggacatcttagtgtagtagacatggaacactatactcgcccagtagcagacatactcgctcgggctgaaggccaatccatcctgttaggacacagcctgggcggcgcctctatctcttggttggcccaacaccacccagacaaagttgctggtctcatttatctgaccgccgttttgacagcgccaggcataacaccggagacattcgttttaccaggcgagccaaatcgtggaacgcctcatgcactcgacttgatccaaccagttgatgagggacggggattgcaggctgatttctcccggttagagagacttcgtgaggtctttatgggcgactaccctggcgaagggatgccgcctgcagagcagtttattcaaacacagtcaacagtgccgtttggtacgcctaatccaatggaggggcgtgctcttgaaattcctcgcctttatatagaagcgcttgacgatgtggtcataccgatagccgtccaacgccaaatgcaaaaggagtttccaggcccggtggctgtcgtatcactcccagctagccacgcaccttactatagtatgccagagagacttgcggaggcaattgccgattttgcagatgccccggccgaatatcgtcagactgccacaaaggcgggccctgatagaccggcaggggcggacggtggccgggctgaccgtgcggatctgccataacaaagcccgaaaggaagctgagttggctgctgccaccgctgagcaataactagcataaccccttggggcctctaaacgggtcttgaggggttttttgctgaaaggaggaactatatccggat